(19) United States
(12) Patent Application Publication
(10) Pub. No.: US 2007/0025888 A1
(43) Pub. Date: Feb. 1, 2007

(54) Diffuser

(76) Inventors: Anil J. Gupte, St. Charles, IL (US); Lanee Reuther, Naperville, IL (US); Juan Vasquez, Bartlett, IL (US)

Correspondence Address: MARSHALL, GERSTEIN & BORUN LLP, 233 S. WACKER DRIVE, SUITE 6300, SEARS TOWER, CHICAGO, IL, 60606, US

(21) Appl. No.: 11/194,860

(22) Filed: Aug. 1, 2005

(51) Int. Cl. A61L 9/03

(52) U.S. Cl. 422/125; 422/124

(57) ABSTRACT

A diffuser includes a heat source, a rotating element, and a fragranced article. The rotating element is disposed above the heat source and has a pivot. The fragranced article includes a fragrance and a fragrance carrier. The fragranced article is associated with the rotating element, such that movement of the rotating element about the pivot according to convection currents produced by the heat source causes the fragrance to be diffused from the fragranced article.

### BACKGROUND

This patent is directed to a diffuser, and in particular to a diffuser that may utilize a heat source that creates convection currents that cause a rotating element to move.

### SUMMARY OF THE INVENTION

In one aspect, a diffuser is provided. The diffuser includes a heat source, a rotating element, and a fragranced article. The rotating element is disposed above the heat source and has a pivot. The fragranced article includes a fragrance and a fragrance carrier. The fragranced article is associated with the rotating element, such that movement of the rotating element about the pivot according to convection currents produced by the heat source causes the fragrance to be diffused from the fragranced article.

In another aspect, another diffuser is provided. The diffuser includes a candle, a fan, and a fragranced article. The fan is disposed above the candle, and has a pivot. The fragranced article includes a fragrance and a fragrance carrier. The fragrance article is coupled to the fan, such that movement of the fan about the pivot according to convection currents produced by the heat source causes movement of the fragranced article and diffusion of the fragrance from the fragranced article.

Additional aspects of the disclosure are defined by the claims of this patent.

### BRIEF DESCRIPTION OF THE DRAWINGS

FIG. 1 is a frontal view of an embodiment of a diffuser according to the present disclosure;

FIG. 2 is a perspective view of a second embodiment of a diffuser;

FIG. 3 is a perspective view of a third embodiment of a diffuser;

FIG. 4 is a perspective view of a fourth embodiment of a diffuser;

FIG. 5 is a perspective view of a fifth embodiment of a diffuser;

FIG. 6 is a bottom plan view of the diffuser of FIG. 5; and

FIG. 7 is a perspective view of a sixth embodiment of a diffuser.

### DETAILED DESCRIPTION OF VARIOUS EMBODIMENTS

Although the following text sets forth a detailed description of numerous different embodiments of the invention, it should be understood that the legal scope of the invention is defined by the words of the claims set forth at the end of this patent. The detailed description is to be construed as exemplary only and does not describe every possible embodiment of the invention since describing every possible embodiment would be impractical, if not impossible. Numerous alternative embodiments could be implemented, using either current technology or technology developed after the filing date of this patent, which would still fall within the scope of the claims defining the invention.

It should also be understood that, unless a term is expressly defined in this patent using the sentence “As used herein, the term ‘______’ is hereby defined to mean . . . ” or a similar sentence, there is no intent to limit the meaning of that term, either expressly or by implication, beyond its plain or ordinary meaning, and such term should not be interpreted to be limited in scope based on any statement made in any section of this patent (other than the language of the claims). To the extent that any term recited in the claims at the end of this patent is referred to in this patent in a manner consistent with a single meaning, that is done for sake of clarity only so as to not confuse the reader, and it is not intended that such claim term be limited, by implication or otherwise, to that single meaning. Finally, unless a claim element is defined by reciting the word “means” and a function without the recital of any structure, it is not intended that the scope of any claim element be interpreted based on the application of 35 U.S.C. §112, sixth paragraph.

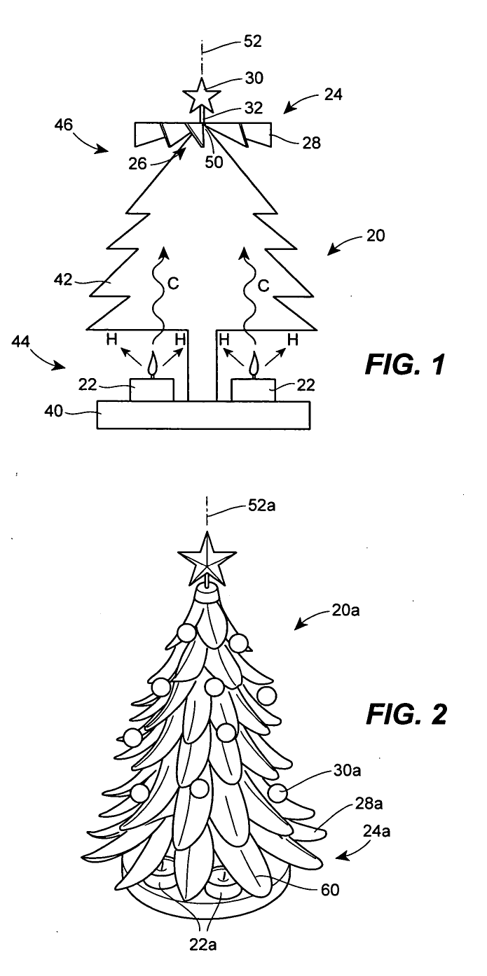

FIG. 1 illustrates an embodiment of a diffuser 20. The diffuser 20 may include a heat source 22. The diffuser 20 may also include a rotating element 24 (which may be a fan or fan-like), the rotating element 24 being disposed above the heat source. The rotating element 24 may have a pivot 26 and a plurality of blades 28 that may be disposed at an angle relative to the horizontal plane. The diffuser 20 may further include a fragranced article 30, which fragranced article 30 may include a fragrance and a fragrance carrier. The fragrance article 30 may be associated with the rotating element 24, such that movement of the rotating element 24 about the pivot 26 according to convection currents (“C”) produced by the heat source 22 causes movement of the fragranced article 30. As shown, the fragranced article 30 is attached to the rotating element 24 by a connecting shaft 32.

The diffuser 20 may have a first mode of operation, wherein the heat source 22 is not active. In this mode of operation, fragrance may still diffuse from the fragranced article 30. In a second mode of operation, the heat sources 22 may be active. The heat sources 22 may generate convection currents in the air. When the convection currents impinge upon the rotating element 24, and in particular the blades 28 of the rotating element 24, the rotating element 24 may move about the pivot 26. The movement of the rotating element 24 about the pivot 26 may cause, in turn, movement of the fragranced article 30, which may cause fragrance to be diffused from the fragranced article. It is believed that the amount of fragrance diffused from the article in the second mode of operation should be greater than in the first mode of operation because of the movement of the fragranced article 30.

Also in the second mode of operation, heat (“H”) from the heat source 22 may impinge upon the fragranced article 30. As a consequence, the fragranced article 30 may diffuse still further fragrance. The fragrance diffused from the fragranced article 30 caused by the heat may assist in making the amount of fragrance diffused in the second mode of operation greater than the first mode of operation. Moreover, even if the rotating element 24 stops while the heat source 22 is active, the heat from the heat source 22 impinging on the fragranced article 30 may cause the fragranced article 30 to diffuse more fragrance in the second mode of operation than in the first mode of operation.

Returning to FIG. 1, the first embodiment of the diffuser 20 is discussed in greater detail. The diffuser 20 further includes a base 40 and a frame 42. The frame 42 may be coupled to the base 40, for example, by attaching a lower end 44 of the frame 42 to the base 40 through the use of an adhesive for example. The pivot 26 is defined, in part, at an upper end 46 of the frame 42. As shown, a surface 48 of the frame 42 defines a point (or bearing) 50 on which the rotating element 24 is balanced. The frame 42 may define a decorative element; for example, the frame 42 shown in FIG. 1 is in the shape of a holiday tree. It will be recognized that the frame 42 may take on a variety of shapes, may be one-, two- or three-dimensional, and need not be symmetrical about multiple axes or even about any axis.

The heat source 22 may be disposed on the base 40 of the diffuser 20. The rotating element 24 is thus disposed above the heat source 22 by the height of the frame 42. The heat source 22, preferably, may be a candle. For example, a tealight may be used if a shorter period of activation of the diffuser 20 is desired, while a votive may be used if a longer period of activation of the diffuser 20 is desired. Alternatively, the heat source 22 may be an oil or liquid-paraffin cartridge, or may be a flameless heat source, such as an electrical bulb.

The rotating element 24 may be disposed on the upper end 46 of the frame 42. Moreover, the plurality of blades 28 that define the rotating element 24 may be angled relative to the horizontal plane, and may also be angled relative to an axis of rotation 52 of the rotating element 24. The angle of the blades 28 may assist in causing the rotating element 24 to move in response to the convection currents caused by the heat source 22. The angle of the blades 28 may also assist in maintaining the rotating element 24 on the pivot 26.

As mentioned above, the fragranced article 30 may be coupled to the rotating element 24 by a connecting shaft 32. In fact, the fragranced article 30 may be attached, by adhesive, for example, to the connecting shaft 32, which may be attached to the rotating element 24 by adhesive as well. It will be recognized that this is not the only manner in which the fragranced article 30 may be coupled to the rotating element 24.

For example, other manners of attachment, mounting, joinder, fastening etc. may be used to couple the fragranced article 30 to the rotating element 24. Additionally, the coupling of the fragranced article 30 to the rotating element 24 may not be permanent; the fragranced article 30 may be removeably or releasably coupled to the rotating element 24. Further, the fragranced article 30 may be coupled directly to the rotating element 24, rather than coupled indirectly to the rotating element 24 via the connecting shaft 32. Moreover, the fragranced article 30 may be formed with the rotating element 24, either in whole or in part. As a further alternative, as discussed in greater detail below, the fragranced article 30 may be disposed about the heat source 22, or may be coupled to a frame that may be disposed about the heat source 22.

As stated previously, the fragranced article 30 may include a fragrance carrier. The carrier may be selected from materials such as waxes, gels, polymers, and ceramics. The non-exhaustive list of waxes may include waxes from natural, synthetic or petroleum sources. The non-exhaustive list of gels may include gels using hydrocarbon oil, vegetable oil, mineral oil, polyamides, polymers, glycols or glycerols. The non-exhaustive list of polymers may include cellulosics, polyethylene, polypropylene, polyvinyl chloride, polyethylene terephthalate, polystyrene, polycarbonate, ABS, EVA, PEBAX and polymethylpentene. Alternatively, materials such as wood, paper, paperboard and silicones may be used.

As also previously stated, the fragranced article 30 may include a fragrance. The fragrance may be selected from the non-exhaustive list of fragrances including musk oil, civet, castreum, ambergris, plant perfumes, sandalwood oil, neroli oil, bergamot oil, lemon oil, lavender oil, sage oil, rosemary oil, peppermint oil, eucalyptus oil, menthol, camphor, verbena oil, citronella oil, cauout oil, salvia oil, clove oil, chamomile oil, costus oil, labdanum oil, broom extract, carrot seed extract, jasmine extract, minmosa extract, narcissus extract, olibanum extract, rose extract, acetophenonene, dimethylinadane derivatives, naphthaline derivatives, allyl caprate, alpha-amylcinnamic aldehyde, anethole, anisaldehyde, benzyl acetate, benzyl alcohol, benzyl propionate, bomeol, cinnmayl acetate, cinnamyl alcohol, citral citronellal, cumin aldehyde, cyclamen aldehyde, decanol, ethyl butyrate, ethyl caprate, ethyl cinnamate, ethyl vanillin, eugenol, geraniol, exenol, alpha-hexylcinnamic aldehyde, hydroxycitrolnellal, indole, iso-amyl acetate, iso amyl iso-valeratek iso-eugenol, linalol, linalyl acetate, p-methylacetophenone, methyl anthranilate, methyl dihydroasmonate, methyl eugenol, methyl-beta-naphthol ketone, methylphenhlcarbinyl acetate, musk ketol, mustk xylol, 2,5,6-nanodinol, gamma-nanolactone, phenylacetoaldehydodimethyl acetate, beta-phenylethyl alcohol, 3,3,5-trimethylcyclohexanol, gamma-undecalactone, undecenal, vanillin, and mixtures thereof.

The fragranced article 30 may be formed from the carrier and the fragrance according to well-known methods.

It will be recognized that more than one fragranced article 30 may be included in the diffuser 20, as is explained in greater detail below. According to such an embodiment, each of a plurality of fragranced articles 30 may have the same fragrance. Alternatively, one or more of the plurality of fragranced articles may have a fragrance that is different than the fragrances of the other of the plurality of fragranced articles. It will be recognized that this may provide the user with a great degree of freedom of choice in this regard.

Similarly, it will be recognized that the heat source 22 may also be fragranced, contain a fragrance or diffuse fragrance. For example, according to one embodiment, the heat source 22 may be a fragranced candle. According to such an embodiment, the fragranced article 30 may have the same fragrance as the heat source 22. This combination may provide an increase in fragrance diffused into the surroundings, or a fragrance “boost.” Alternatively, the fragranced article 30 may have a different fragrance than that of the heat source 22. This combination may provide a pleasing blend of fragrances diffused into the surroundings, or a fragrance “fusion.”

In addition, the materials which may be used to make the frame 40, the base 42, and the rotating element 24 discussed above may be selected from a variety of materials. A non-exhaustive list of such materials may include metals, plastics, resins, glass, fiberglass, ceramics and wood, with the selection of the material selected being recognized by one skilled in the art. A non-exhaustive list of the metals (which may include plated finishes) may include steel (which may be polished, brushed, antiqued, satin finished, embossed, etc.), aluminum (which may be polished, brushed, antiqued, satin finished, galvanized, etc.), iron (which may be wrought, antiqued, etc.), nickel (which may be brushed, antiqued, polished, etc.), brass (which may be polished, antiqued, etc.), gold, silver, pewter, and chrome. A non-exhaustive list of the plastics (which may include copolymers and multi-layer composites) may include acrylonitrile butadiene styrene, high density polyethylene, linear low density polyethylene, low density polyethylene, polyvinyl chloride, polypropylene, polyvinylidiene chloride, polyester teraphthalate, polycarbonate, polystyrene, high impact polystyrene, ethylene ethyl acrylate, ethylene vinyl acetate, polyurethane (which may be cold cast, thermoset, etc.), urea formaldehyde, melamine formaldehyde, phenol formaldehyde and polyesters. A non-exhaustive list of ceramics (which may include glazed and unglazed materials) may include dolomite, stoneware, terra cotta, and porcelain. These materials may be tinted, painted or coated with a variety of materials.

Alternative embodiments of the diffuser 20 are now discussed below relative to FIGS. 2-4. Similar elements are numbered similarly, with like elements of different embodiments distinguished through the addition of a lower-case letter.

FIG. 2 illustrates a diffuser 20a. The diffuser 20a, like the diffuser 20, may include a heat source 22a (in this case a plurality of tealights), a rotating element 24a, and a plurality of fragranced articles 30a. Unlike the diffuser 20, the diffuser 20a may include a base 40a, but no frame. Instead, according to this embodiment, the rotating element 24a (which also defines a decorative element, in this case a holiday tree) may include a shaft 60 that is pivotally coupled to the base 40a. When the heat source 22a is activated, the convection currents may impinge upon the blades 28a (which are shaped in the form of leaves or boughs of a tree), causing the rotating element 24a to move about its axis of rotation 52a. The movement of the rotating element 24a may cause movement of the plurality of fragranced articles 30a, in turn causing the fragranced articles 30a to diffuse fragrance into the surroundings.

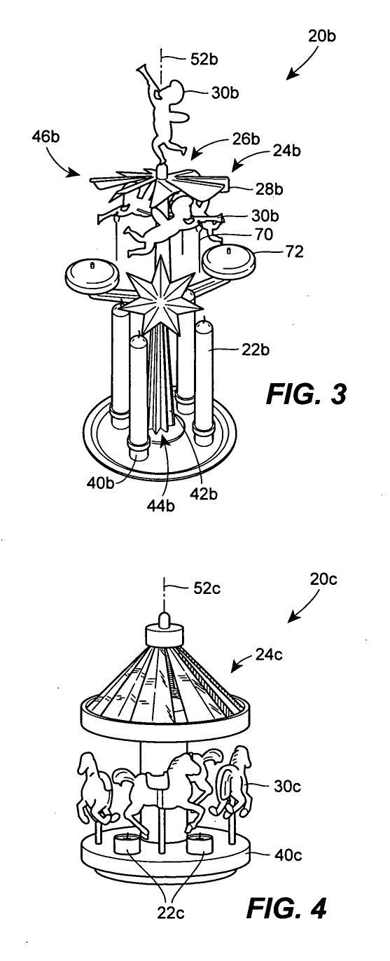

FIG. 3 illustrates a diffuser 20b. The diffuser 20b may also include a heat source 22b (in this case, tapered candles), a rotating element 24b, and a plurality of fragranced articles 30b. Unlike the diffusers 20, 20a, some of the fragranced articles 30b of the diffuser 20b may be attached to edges of the blades 28. According to this embodiment, clappers 70 may be coupled to the fragranced articles 30b, which clappers 70 cooperate with bells 72 to create sounds (chimes) at the same time as fragrance is diffused from the fragranced articles 30b.

FIG. 4 illustrates a diffuser 20c. The diffuser 20c may also include a heat source 22c (in this case, votive candles), a rotating element 24c, and a plurality of fragranced articles 30c. Unlike the diffusers 20, 20a, 20b, the fragranced articles 30c of the diffuser 20c are disposed about the rotating element 24c, but do not rotate with the rotating element 24c. Instead, the fragranced articles 30c (which are in the form of carrousel horses) are disposed about the rotating element 24c and the heat source 22c. It will be recognized that according to other embodiments, the fragranced articles 30c may be coupled to the rotating element 24c in a fashion similar to that shown in the embodiment of FIG. 3.

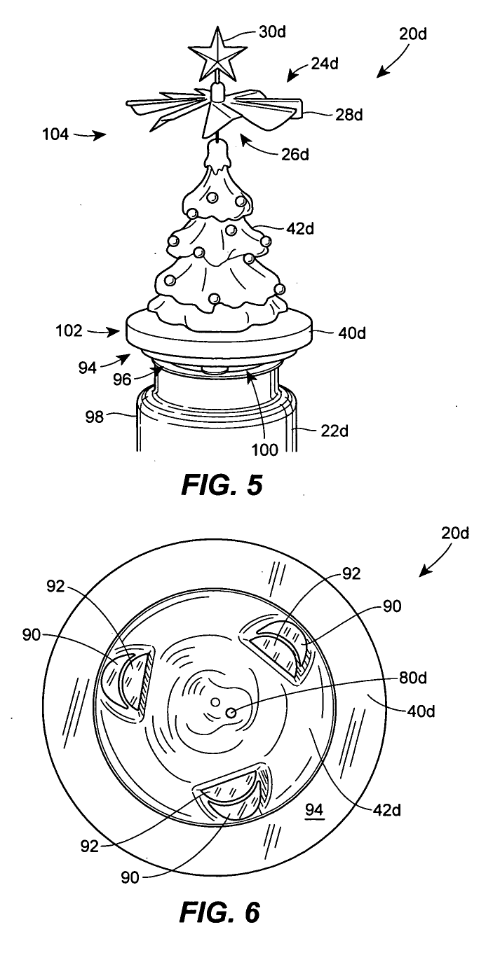

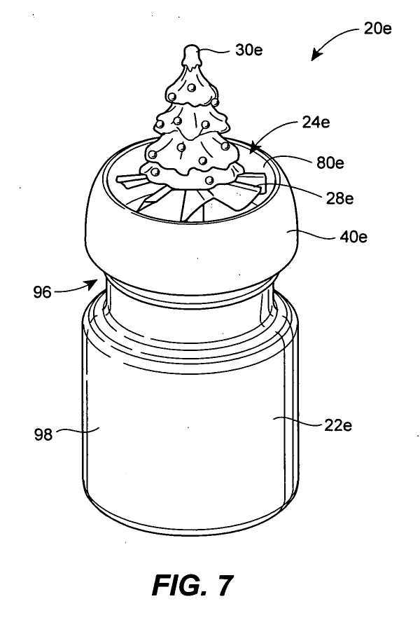

A further embodiment of the diffuser 20d is shown in FIGS. 5 and 6, with a similar embodiment of the diffuser 20e shown in FIG. 7. In the embodiments of the diffuser 20d, 20e shown in FIGS. 5-7, the diffuser 20d, 20e may be disposed above a heat source 22d, 22e in the form of an apothecary jar candle, although any candle disposed within a container may be substituted therefore. In particular, the base 40d, 40e of the diffuser 20d, 20e may be disposed on the container (or jar) of the heat source 22d, 22e, and in particular on a rim of the container that defines an opening thereto. The convection currents from the heat source 22d. 22e may pass through passages or openings 80d, 80e in the base or the frame of the diffuser 20d, 20e, and thereby come into contact with the blades 28d, 28e of the rotating element 24d, 24e.

In particular, the diffuser 20d of FIGS. 5 and 6, the base 40d may have a plurality of spacers 90 and a plurality of tabs 92 disposed on a first side 94 thereof (see FIG. 6). The base 40d may be disposed on a rim 96 of a container (in this case, a jar) 98 in which a heat source (in this case, a candle) 22d is disposed. The plurality of tabs 92, which may be disposed at regular intervals in a pattern on the first side 94 of the base 40d, may center the base 40d on the container 98, and thus, hopefully, on the heat source 22d. The spacers 90 may abut the rim 96 of the container 98 such that a space or passage 100 is defined between the rim 96 of the container 98 and the first side 94 of the base 40d. This space or passage 100 may allow air to enter the container 98 during combustion of the candle 22d.

The frame 42d of the diffuser 20d may depend from a second side 102 of the base 40d. The rotating element 24d may be supported on a pivot 26d positioned at an upper end 104 of the frame 42d. The passages or openings 80d in the frame 42d may permit convection currents, which may be established by the combustion of the candle 22d, to pass upwards and contact the blades 28d of the rotating element 24d. Movement of the rotating element 24d may cause movement of the one or more fragranced articles 30d (in this case, in the form of a star), which in turn may cause fragrance to be diffused from the fragranced article 30d. Heat from the heat source 20d may also cause fragrance to be diffused from the fragranced article 30d.

As stated above, the diffuser 20e shown in FIG. 7 is similar to the diffuser 20d shown in FIGS. 5 and 6. That is, both the diffuser 20d and the diffuser 20e may be disposed on a rim 96 of a container 98, and may include spacers that may assist in defining passages and tabs that may assist in centering the diffuser 20e on the container 98. However, diffuser 20e differs in that there is no frame, the passage 80e is defined in the base 40e, and the rotating element 26e is mounted in the passage 80e, by mating bearings or bearing surfaces, for example. Otherwise, the operation of the diffuser 20e is similar to that of diffuser 20d, or any of the diffusers 20, 20a, 20b, 20c discussed above.

What is claimed is:

1. A diffuser, comprising:
a heat source;
a rotating element disposed above the heat source, the rotating element having a pivot;
a fragranced article comprising a fragrance and a fragrance carrier,
the fragrance article associated with the rotating element, movement of the rotating element about the pivot according to convection currents produced by the heat source causing the fragrance to be diffused from the fragranced article.

2. The diffuser according to claim 1, wherein the heat source comprises a candle.

3. The diffuser according to claim 2, wherein:
the candle comprises a fragrance, and
the fragrance of the candle and the fragrance of the fragranced article are the same.

4. The diffuser according to claim 2, wherein:
the candle comprises a fragrance, and
the fragrance of the candle and the fragrance of the fragranced article are the different.

5. The diffuser according to claim 1, wherein the fragranced article is coupled to the rotating element.

6. The diffuser according to claim 5, wherein the fragranced article is attached to the rotating element.

7. The diffuser according to claim 1, wherein the fragranced article is disposed about the rotating element.

8. The diffuser according to claim 1, further comprising a frame disposed about the rotating element, the fragranced article coupled to the frame.

9. A diffuser, comprising:
a candle;
a fan disposed above the candle, the fan having a pivot; and
a fragranced article comprising a fragrance and a fragrance carrier,
the fragrance article coupled to the fan, movement of the fan about the pivot according to convection currents produced by the heat source causing movement of the fragranced article and diffusion of the fragrance from the fragranced article.

10. The diffuser according to claim 9, wherein the fragranced article is attached to the fan.

11. The diffuser according to claim 9, wherein:
the candle comprises a fragrance, and
the fragrance of the candle and the fragrance of the fragranced article are the same.

12. The diffuser according to claim 9, wherein:
the candle comprises a fragrance, and
the fragrance of the candle and the fragrance of the fragranced article are the different.

13. The diffuser according to claim 9, wherein:
the fragranced article is removeably attached to the fan.

14. The diffuser according to claim 9, comprising:
a base; and
a frame attached to the base,
the candle disposed on the base and the fan disposed on the frame.

15. The diffuser according to claim 9, comprising a base, the fan being pivotally coupled to the base.

16. The diffuser according to claim 9, wherein the candle is disposed in a container having an opening defined by a rim, the diffuser further comprising a base, the base disposed on the rim of the container and the fan disposed on the base.

17. The diffuser according to claim 16, comprising a frame, the frame attached to the base and the fan disposed on the frame.

18. The diffuser according to claim 17, wherein the frame has an opening defined therethrough and the convection currents pass through the opening.

19. The diffuser according to claim 16, wherein the base has an opening defined therethrough, the fan is disposed in the opening, and the convection currents pass through the opening.
